(19) United States
(12) Patent Application Publication
(10) Pub. No.: US 2019/0192046 A1
(43) Pub. Date: Jun. 27, 2019

(54) METHOD AND SYSTEM FOR SELECTING AN INHALER

(71) Applicant: Medituner AB, Stockholm (SE)

(72) Inventor: Henrik LJUNGBERG, Stockholm (SE)

(73) Assignee: Medituner AB, Stockholm (SE)

(21) Appl. No.: 16/325,510

(22) PCT Filed: Aug. 21, 2017
(86) PCT No.: PCT/EP2017/070992

§ 371 Date: Feb. 14, 2019

(30) Foreign Application Priority Data
Aug. 22, 2016 (GB) 1614304.2

(51) Int. Cl. A61B 5/087; G16H 50/20; G16H 40/63; G16H 20/10; A61B 5/00

(52) U.S. Cl. CPC A61B 5/087; G16H 50/20; A61B 5/0022; G16H 20/10; A61B 5/742; G16H 40/63

(57) ABSTRACT

Disclosed is a method for selecting a suitable inhaler device to a user. The method comprises using a spirometer and a computer comprising a display. The method comprising the following steps: A) connecting the spirometer and the computer; B) the computer, or spirometer, indicating to the user to inhale through the spirometer; C) the spirometer sending flow over time data of the inhalation made to the computer; and D) the computer comparing the flow over time data with dose emission over time data for a range of inhalers and indicating the suitable inhaler from the range of inhalers as the inhaler with the largest dose emission within the flow over time data. Disclosed is also a system for establishing a suitable inhaler device. The system comprises a spirometer and a computer connectable to each other for data transfer. The spirometer or the computer is configured to indicate to the user to inhale through the spirometer. The spirometer is configured to send flow over time data of the inhalation made to the computer. The computer is configured to compare the flow over time data with dose emission over time data for a range of inhalers and indicating the suitable inhaler of the range of inhalers as the inhaler with the largest dose emission within the flow over time data.

### TECHNICAL FIELD

The present disclosure relates to a method and system for selecting an inhaler device. More particularly, the present disclosure relates to a method and system to select the most suitable inhaler for a user.

### BACKGROUND

In recent years attention has been turned to inhaler devices. An inhaler is a medical device designed to deliver pharmaceutical drugs into a patients lungs. An inhaler is normally prescribed by a doctor to a user and the user uses the inhaler in a manner that has been prescribed by the doctor. While this provides satisfactory results in everyday life, it would be desirable to select the most suitable inhaler for a user.

Different users inhale differently. Different inhalers emit their dosage differently. For example a metered dose inhaler (MDI) is a type of aerosol that is emitted from the device, while a dry powder inhaler (DPI) contains drug in the form a dry powder that is released as the patient inhales. There are also different subtypes of these medical devices. As a result the amount of effective medication that a user actually inhales can vary very much.

A further problem is that a user may not know how to use the inhaler to get an effective dosage. A user may be anything from a very young child to a very old and senile person. A very young or old person may have problems inhaling medications and it is difficult to know how much, if any, medication they inhale. A user may be active, for example doing sports, and can inhaler very forcefully. A user may not be active at all and/or may have further complications such as lung or neurological disease and may not be able to inhaler very well. With this in mind it is difficult for a user to know how efficient he is using his inhaler and how much of the medication he actually receives with an inhalation.

A further problem is how to provide a solution that is practical and can be used in real life. Furthermore, it is a problem how to do this as cost effective and as reliable as possible. A solution must also be simple and not cumbersome to a user. Additionally, any solution must also be attractive for a user to use. The present invention is directed to overcoming one or more of the problems as set forth above.

### SUMMARY

It is an object of the present invention to provide a method and a system to select the most suitable inhaler for a user. This object can be achieved by the features of the independent claims. Further enhancements are characterized by the dependent claims. A further object may be to provide a method and system where a user may improve his inhalation techniques for a specific inhaler.

According to one embodiment, a method for selecting a suitable inhaler to a user is disclosed. The method comprises using a spirometer and a computer, one or both comprising a display. The method comprises the following steps: A) connecting the spirometer and the computer; B) the computer, or spirometer, indicating to the user to inhale through the spirometer; C) the spirometer sending flow over time data of the inhalation made to the computer; and D) the computer comparing the flow over time data with dose emission over time data for a range of inhalers and indicating the suitable inhaler from the range of inhalers as the inhaler with the largest dose emission within the flow over time data.

In one embodiment, the method further comprises, or selected instead of step D) by the user, the following step: E) displaying the dose emission over time data of an inhaler of the range of inhalers, and displaying the flow over time data of the inhalation on the display by the computer. In one embodiment, the inhaler in step E) is the suitable inhaler indicated in step D).

In one embodiment, the flow over time data is displayed on the display and/or the suitable inhaler is indicated on the display.

In one embodiment, a message is given by the computer to the user how to inhale to get the flow over time data of an inhalation to suit the dose emission over time data of an inhaler of the range of inhalers.

In one embodiment, the computer is one of the following group: a mobile phone, a mobile computer, a watch, a tablet, a PC, or a cloud service.

In one embodiment, the flow of an inhalation is displayed on the display after the inhalation.

In one embodiment, flow resistance of the inhaler is simulated by adjusting flow through a mouth piece of the spirometer.

In one embodiment, a system for establishing a suitable inhaler for a user is disclosed. The system comprises a spirometer and a computer connectable to each other for data transfer. The spirometer or the computer is configured to indicate to the user to inhale through the spirometer. The spirometer is configured to send flow over time data of the inhalation made to the computer. The computer is configured to compare the flow over time data with dose emission over time data for a range of inhalers and indicating the suitable inhaler of the range of inhalers as the inhaler with the largest dose emission within the flow over time data.

In one embodiment, the computer is further configured to display on a display the dose emission over time data of an inhaler of the range of inhalers and the flow over time data of the inhalation.

In one embodiment, the computer is further configured to display on a display flow over time data and/or the suitable inhaler.

In one embodiment, the computer is further configured to display on a display a message to a user how to inhale to get the flow over time data of an inhalation to suit the dose emission over time data of an inhaler of the range of inhalers.

In one embodiment, the computer is one of the following group: a mobile phone, a mobile computer, a watch, a tablet, a PC, or a cloud service.

In one embodiment, the computer is further configured to display on a display the flow of an inhalation after the inhalation.

In one embodiment, the spirometer comprises a mouthpiece, the mouth piece comprising an adjustment of the flow through the mouth piece by adjusting the mouth piece, or changing the mouth piece, or adding an adaptor.

In one embodiment, at least one embodiment of the method uses at least one embodiment of the system to determine a suitable inhaler and/or for training the user to inhale.

At least one of the above embodiments provides one or more solutions to the problems and disadvantages with the background art. Other technical advantages of the present disclosure will be readily apparent to one skilled in the art from the following description and claims. Various embodiments of the present application may obtain only a subset of the advantages set forth. No one advantage is critical to the embodiments. Any claimed and/or described embodiment herein may be technically combined with any other claimed and/or described embodiment(s) herein.

### BRIEF DESCRIPTION OF THE DRAWINGS

The accompanying drawings illustrate presently preferred exemplary embodiments of the disclosure, and together with the general description given above and the detailed description of the preferred embodiments given below, serve to explain, by way of example, the principles of the disclosure.

FIG. 1 is a diagrammatic illustration of exemplary embodiments of the present disclosure;

FIG. 2 is a diagrammatic illustration of different inhalation flows over time;

FIG. 3 is a diagrammatic illustration of dose emission and inhalation flow over time; and

FIG. 4 is a diagrammatic illustration of an exemplary embodiment of the method.

### DETAILED DESCRIPTION

FIG. 1 illustrated an exemplary system comprising a spirometer 20 and a computer 10. The computer 10 may have a display 12. The computer may for example be a smart phone, a tablet, a mobile device, or a PC. The computer 24 may alternatively, or also, be part of the spirometer 20. The spirometer 20 may have a mouth piece 22 to inhale or blow in. The mouth piece 22 can be changed to different types of mouth pieces 22. Hereby the flow resistance in the mouth piece 22 can be changed. In one embodiment, the one and the same mouth piece 22 may be adjustable to adjust the flow resistance through the mouth piece. When a user inhales, or blows, into the mouth piece 22 then the spirometer measures the flow through the spirometer 20. This flow is normally expressed as volume per time. The spirometer 20 may also comprise a computer 24 and/or a display 26. The spirometer 20 and the computer 10 are connected, symbolised with arrow 21 in FIG. 1. The connection 21 may be wireless or wired, and the connection 21 may allow information and/or energy, such as electricity, to be transferred between the computer 10 and the spirometer 20.

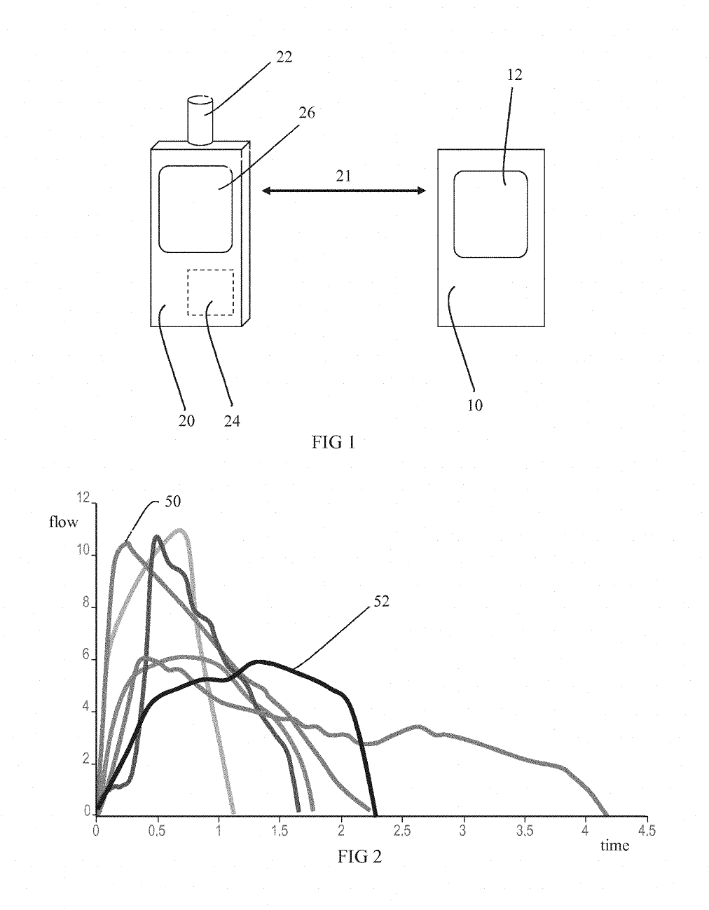

FIG. 2 is a diagrammatic illustration of inhalation flow over time. FIG. 2 shows the inhalation flow on the vertical axis and the time on the horizontal axis. The inhalation flow is normally measured as volume per time, for example litres per minute. The time is normally measured in seconds. Different users inhale differently over time and the different curves show the inhalation flow of different users over time. For example, one user may inhale strongly in the beginning, creating a high flow within the, for example, first second. Such a user may for example have a lower flow later on. This is illustrated by the exemplary curve 50 in FIG. 2. For example, another user may inhale poorly in the beginning, creating a low flow within the first second, but increase the flow as the time goes and then subsequently lower the inhalation flow. This is illustrated by the exemplary curve 52 in FIG. 2. The other exemplary curves illustrate different amounts of inhalation flow during the time of an inhalation.

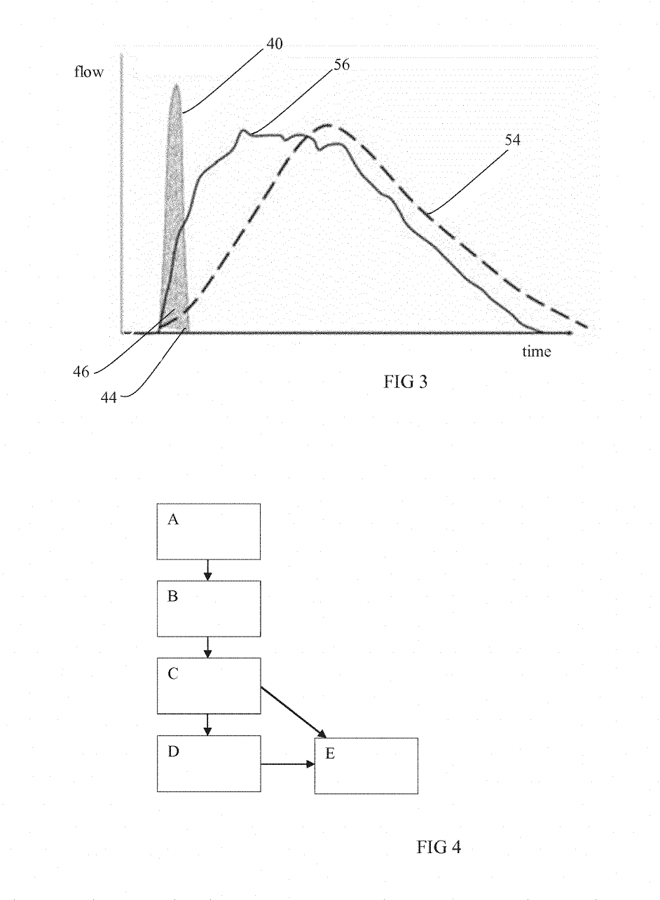

FIG. 3 is a diagrammatic illustration of inhalation flow over time. Just as in FIG. 2, FIG. 3 shows the inhalation flow on the vertical axis and the time of the inhalation on the horizontal axis in a coordinate system. An exemplary dose emission 40 is illustrated and the grey area of the dose emission represents the release of medication by an inhaler over time. Different inhalers may produce different dose emissions 40. For example, a different inhaler may start realising the dose earlier or later, and the dose emission may last longer or shorter. For example, the dose emission of a different inhaler may be larger or smaller. An exemplary curve 54 illustrated how a first user may inhale when using the inhaler and an exemplary curve 56 illustrated how a second user may inhale when using the inhaler. An area 44 illustrates the amount of medication that the first user would receive from using the inhalator. The area 44 is the area of the dose emission 40 under the curve 54 of the first user. An area 46 illustrates the amount of medication that the second user would receive from using the inhalator. The area 46 is the area of the dose emission 40 under the curve 56 of the second user. In FIG. 3, it so happens that the area 46 includes the area 44. The second user with the curve 56 will receive more medication from the inhaler because his inhalation flow over time is more in line with dose emission than the first user with the curve 54. In other words, the inhaler is more suitable to the second user producing the curve 56 then to the first user producing the curve 54. This may also be taken directly from FIG. 3. When comparing the flow over time data 54 and 56 of the users with dose emission over time data 40 for an inhaler or a range of inhalers, the most suitable inhaler is the inhaler with the largest dose emission within the flow over time data. That is the inhaler and user inhalation curve that renders the largest area 44, 46 of dose emission. By comparing inhalation flow over time of a specific user with dose emission of different inhalers the most suitable inhaler for administrating medication to that specific user can be determined. The most suitable inhaler for the specific user would be the inhaler that provides the largest dose emission within the inhalation flow over time of the specific user. In other words, the most suitable inhaler for the specific user would be the inhaler that provides the largest dose emission below, within, the inhalation flow over time curve of the specific user.

In one embodiment, the inhalation flow over time curve of a specific user may be an average of two or more inhalations. An exemplary curve 56 in FIG. 3 may be an average curve of a plurality of inhalations. The plurality of inhalations can be taken in a row over a short time period, such as for example 60 seconds. The plurality of inhalations can be an average over several days. The plurality of inhalations can be, substantially, all inhalations made by one and the same user on the same spirometer or on different equipment with the computer configured to calculate the average. In one embodiment, the user may be able to reset the average.

According to one embodiment, a method for selecting a suitable inhaler to a user is disclosed. The method comprises using a spirometer 20 and a computer 10. The spirometer 20 and/or the computer 10 comprises a display 12, 26. The method comprises the following steps that may be taken in any order, as long as it makes technical sense. FIG. 4 is a diagrammatic illustration of an exemplary embodiment of the method.

Step A), connecting the spirometer 20 and the computer 10. This may be done by a wireless connection 21 and/or a wired connection 21. The connection allows data to be transferred between the spirometer 20 and the computer 10.

Step B), the computer 10 and/or the spirometer 20 indicates to the user to inhale (or exhale) through the spirometer. The computer 10 and/or the spirometer 20 is configured to give an indication to inhale or exhale. This indication may be a visible sign on the display 12 and/or 26, and/or an audio signal, and/or a vibration.

Step C), the spirometer 20 sends flow over time data of the inhalation made by the user to the computer 10. It may be one or more inhalations. The spirometer 10 is configured to send inhalation flow data or exhalation flow data and the corresponding time via the connection 21 to the computer 10.

Step D), the computer 10 compares the flow over time data with dose emission over time data for a range of inhalers. The computer 10 indicates the suitable inhaler from the range of inhalers as the inhaler with the largest dose emission 40 within the flow over time data 54, 56. The computer 10 is configured to indicate the suitable inhaler, as also explained above with reference to FIG. 3. In other words, the suitable inhaler is the inhaler with the largest dose emission 40 covered by the inhalation (the inhalation flow curve over time data). The suitable inhaler is the inhaler which can deliver the maximum of dose emission 40 from the inhaler into the patient during the inhalation.

According to at least one embodiment, the method may further comprise the following step:

E) displaying the dose emission over time data of an inhaler of the range of inhalers, and displaying the flow over time data of the inhalation on the display by the computer 10. For example, the display 12 or 26 may display what is basically shown in FIG. 3, but with only one of the curves 54 or 56. In this way a user can see how he has inhaled (or exhaled) with reference to the dose emission. The user can then practice and improve his inhalation flow to better fit the dose emission. For example, if he inhales according to the inhalation flow curve 54, then he may try to inhale more in the start and thereby come to the inhalation flow curve 56. As a result, the user would be able to take in more of the dose emission 40.

According to at least one embodiment, the described step E) may be made instead of step D). For example, the user may decide to practice his inhalations. He can then select the MDI he uses and gets the dose emission 40 for that MDI. By inhaling he may then see how his inhalation flow over time data curve 56 is. As both curves (dose emission and inhalation flow) are displayed on the screen 12 or 26, a technical effect is that the user can train to inhale as much as possible of the dose emission of a specific inhaler, for example an inhaler that may be more suitable for other reasons, such as for example availability.

In at least one embodiment, the inhaler in step E) is the suitable inhaler indicated in step D). The suitable inhaler as indicated by the method may be the inhaler who's dose emission the user can practice against.

In at least one embodiment, a message is given by the computer 10 or 24 to the user how to inhale to get the flow over time data 54 or 56 of an inhalation to suit the dose emission over time data 40 of an inhaler of the range of inhalers. The message may be an indication, such as guidance or instructions. This may be in the form of text or illustrations or audio. This allows the user to get instructions how to change his inhalation to inhale more of the dose emission 40. For example the message could be “inhale stronger from the start”. The inhaler may be the inhaler in step E) or the suitable inhaler of step D).

In at least one embodiment, the flow over time data is displayed on the display 12, 26. In addition, or as an alternative, the suitable inhaler is indicated on the display. This indication may be a picture of the suitable inhaler and/or its name and/or product identification. In addition hereto the dose emission may be displayed.

In at least one embodiment, the computer 10 is one of the following group: a mobile phone, a mobile computer, a watch, a tablet, a PC, or a cloud service. According to one embodiment, the computer 10 may be integrated with the spirometer 20 as indicated with reference 24 in FIG. 1.

In at least one embodiment, the flow of an inhalation is displayed on the display 12 or 26 after the inhalation. Subsequently to the inhalation, the display may display the inhalation flow on the display 12 or 26. That is, the display displays only the flow instead of the flow over time. The suitable MDI can be selected by only showing the inhalation flow instead of the inhalation flow over time. In one embodiment, the inhalation flow is only displayed if the user selects the flow to be displayed.

In one embodiment a system for establishing a suitable inhaler is disclosed. An exemplary embodiment of the system is diagrammatically illustrated in FIG. 1. The system comprises a spirometer 20 and a computer 10 (and/or 24) connectable to each other for data transfer 21. The spirometer 20 or the computer 10 is configured to indicate to the user to inhale through the spirometer. The spirometer 20 is configured to send flow over time data of the inhalation made to the computer 10, 24. The computer 10, 24 is configured to compare the flow over time data 54, 56 with dose emission over time data 40 for a range of inhalers and indicating the suitable inhaler of the range of inhalers as the inhaler with the largest dose emission 46 within the flow over time data.

In at least one embodiment, the computer 10, 24 is further configured to display on a display 12, 26 the dose emission over time data 40 of an inhaler of the range of inhalers and the flow over time data 54, 56 of the inhalation. The computer 10 may be configured to put them together in the same view in a coordinate system with inhalation flow on the y-axis and time on the x-axis. This would display the dose emission 46, 44 that the user would theoretically inhale, as indicated in FIG. 3.

In at least one embodiment, the computer 10, 24 is further configured to display on a display 12, 26 flow over time data and/or the suitable inhaler. The computer may be configured to display the suitable inhaler as a picture, and/or by name, and/or by product number.

In at least one embodiment, the computer 10, 24 is further configured to display on a display 12, 26 a message, to the user, how to inhale to get the flow over time data 54, 56 of an inhalation to suit the dose emission over time data 40 of an inhaler of the range of inhalers. The message may be instructions in the form of one or more of illustrations, movies, text, or voice. In this way a user is instructed how to change his inhalation profile.

In at least one embodiment, the computer is one of the following group: a mobile phone, a mobile computer, a watch, a tablet, a PC, or a cloud service. Preferably it is a personal handheld electronic device of the user.

In at least one embodiment, the computer 10, 24 is further configured to display on a display 12, 26 the flow of an inhalation after the inhalation. The computer may be configured to display the flow of the inhalation, without displaying the flow over time. The display of the flow may be displayed in real time. The display of flow may be displayed as a number, for example 30 l/min. An inhaler may be selected as suitable only based on the flow, instead of the flow over time.

In at least one embodiment, one or more embodiments of the method may use one or more embodiments describing the system to determine a suitable inhaler and for training the user to inhale.

In at least one embodiment, the method and/or system further comprises data for a range of inhalers. This data comprises dose emission data for each inhaler and identification data for each inhaler. The identification data of the inhaler may comprise an illustration of the inhaler, or the inhaler name, or product identification, for example a number. Alternatively, or in addition, the method and/or system is further configured to download inhaler data.

In at least one embodiment, the computer 10 is a mobile handheld device and is configured to select a suitable inhaler by having an application installed. This allows the method and system to use already existing equipment and only a spirometer 20 must be provided for determining a suitable inhaler.

In at least one embodiment, the inhalation flow over time may be displayed in real time in a coordinate system with, or without, dose emission data.

In at least one embodiment, the method and/or system further comprises testing a user's inhalation using different resistance to inhalation. The mouth piece 22 on the spirometer may be configured to have a specific resistance. Different mouth pieces 22 may have different resistance. By changing to different types of mouth pieces 22, the inhalation, or exhalation, resistance of the spirometer is changed. Hereby different types of inhalers may be simulated. In one embodiment, the one and the same mouth piece 22 may be adjustable to adjust the flow resistance through the mouth piece. In one embodiment, the inhalation, or exhalation, resistance may be adjusted by using the actual inhaler device itself, or at least parts thereof. In this case the inhaler may be connected using an adaptor to the spirometer 20. The inhalation is then carried out through the actual inhaler as well as the spirometer.

According to at least one embodiment, the system may provide reports or logged information for the benefit of treating medical staff or the patient him or herself regarding the patient's condition as well as compliance to medication.

According to at least one embodiment, the system may link with the patient's medical records giving treating medical staff information about the patient's condition and making dosage adjustments improving treatment.

It will be apparent to those skilled in the art that various modifications and variations can be made to the system and dosage regime. Especially that one or more of the embodiments disclosed above can be combined with each other. Other embodiments will be apparent to those skilled in the art from consideration of the specification and practice of the disclosed system and dosage regime. It is intended that the specification and examples be considered as exemplary only, with a true scope being indicated by the following claims and their equivalents.

1. A method for selecting a suitable inhaler to a user, the method comprising:
A) connecting a spirometer and a computer, the computer comprising a display;
B) the computer, or spirometer, indicating to the user to inhale through the spirometer;
C) the spirometer sending flow over time data of the inhalation made to the computer; and
D) the computer comparing the flow over time data with dose emission over time data for a range of inhalers and indicating the suitable inhaler from the range of inhalers as the inhaler with the largest dose emission within the flow over time data.

2. The method according to claim 1, comprising:
E) displaying the dose emission over time data of an inhaler of the range of inhalers, and displaying the flow over time data of the inhalation on the display by the computer.

3. The method according to claim 2, wherein the inhaler in step E) is the suitable inhaler indicated in step D).

4. The method according to claim 1, wherein the flow over time data is displayed on the display and/or the suitable inhaler is indicated on the display.

5. The method according to claim 1, comprising that a message is given by the computer to the user how to inhale to get the flow over time data of an inhalation to suit the dose emission over time data of an inhaler of the range of inhalers.

6. The method according to claim 1, wherein the computer is one of the following: a mobile phone:
a mobile computer;
a watch;
a tablet;
a PC; and
a cloud service.

7. The method according to claim 1, comprising that the flow of an inhalation is displayed on the display after the inhalation.

8. The method according to claim 1, wherein flow resistance of the inhaler is simulated by adjusting flow through a mouth piece of the spirometer.

9. A system for establishing a suitable inhaler, the system comprising:
a spirometer;
a computer connectable for data transfer;
wherein the spirometer or the computer is configured to indicate to the user to inhale through the spirometer;
wherein the spirometer is configured to send flow over time data of the inhalation made to the computer; and
wherein the computer is configured to compare the flow over time data, with dose emission over time data for a range of inhalers and indicating the suitable inhaler of the range of inhalers as the inhaler with the largest dose emission within the flow over time data.

10. The system according to claim 9, wherein the computer is configured to display the dose emission over time data of an inhaler of the range of inhalers and the flow over time data of the inhalation.

11. The system according to claim 9, wherein the computer is configured to display at least one of flow over time data and the suitable inhaler.

12. The system according to claim 9, wherein the computer is configured to display on a display a message to a user how to inhale to get the flow over time data of an inhalation to suit the dose emission over time data of an inhaler of the range of inhalers.

13. The system according to claim 9, wherein the computer is one of the following:
a mobile phone;
a mobile computer;
a watch;
a tablet;
a PC; and
a cloud service.

14. The system according to claim 9, wherein the computer is configured to display the flow of an inhalation after the inhalation.

15. The method according to claim 1, comprising, instead of step D), responsive to selection by the user:
E) displaying the dose emission over time data of an inhaler of the range of inhalers, and displaying the flow over time data of the inhalation on the display by the computer.
